(19) United States
(12) Patent Application Publication
(10) Pub. No.: US 2007/0042493 A1
(43) Pub. Date: Feb. 22, 2007

(54) METHOD AND PROCESS OF GENETIC TRANSFORMATION USING SUPERCRITICAL FLUIDS

(76) Inventors: Ali O. Kilic, Chicago, IL (US); James T.C. Yuan, Naperville, IL (US)

Correspondence Address: Linda K. Russell, Air Liquide Intellectual Property Dept., 2700 Post Oak Blvd., Suite 1800, Houston, TX, 77056, US

(21) Appl. No.: 11/424,197

(22) Filed: Jun. 14, 2006

Related U.S. Application Data
(60) Provisional application No. 60/710,132, filed on Aug. 22, 2005.

(51) Int. Cl. C12N 15/09; C12N 15/74

(52) U.S. Cl. 435/455; 435/471

(57) ABSTRACT

Methods are provided for improving the ability of non-naturally transformable cells to take up and integrate DNA. Generally, the invention provides a method for transforming cells, comprising combining deoxyribonucleic acid (DNA) and a recipient cell culture for the uptake of the DNA, placing mixture of recipient cell culture and DNA into a vessel, injecting a supercritical fluid into the vessel, removing the recipient cells from the vessel, and placing removed cells into a growth media with selective conditions to allow expression of transformed DNA.

### CROSS-REFERENCE TO RELATED APPLICATIONS

This application claims the benefit under 35 U.S.C. § 119(e) to provisional application No. 60/710,132, filed Aug. 22, 2005, the entire contents of which are incorporated herein by reference.

### BACKGROUND

1. Field of the Invention

The invention relates to the improvement of genetic transformation efficiency in cells, more specifically to a method for affecting the ability of non-naturally transformable cells to take up and integrate extracellular DNA.

2. Description of the Related Art

One of the greatest strengths of molecular biology is the ability to manipulate genetic material of an organism through genetic engineering. A genetically engineered organism is an organism whose genetic material has been altered using techniques generally known as recombinant deoxyribonucleic (DNA) technology. Recombinant DNA technology allows one to combine DNA molecules from different sources and incorporate them into one molecule in the laboratory. This newly formed DNA molecule can then be inserted and incorporated into the genome of another organism that doesn't naturally carry the newly added DNA. Thus, the phenotype of the organism, or the proteins it produces, can be modified through the manipulations of its DNA, or genes.

Genetic transformation is a common method of modifying the genetic material in living cells. It is a process in which foreign DNA is taken up by cells from their surrounding environment and incorporated into their genome. The transformation mechanisms rely on the cells ability to uptake and stably maintain the extracellular DNA, which can be genomic, plasmid, bacteriophage, or any other natural or artificial DNA. Cells can uptake DNA as plasmids: self-replicating circles of DNA that usually contain a selectable marker gene such as a drug resistance gene.

Some bacterial strains naturally develop competence (the ability to take up and integrate extracellular DNA) under certain environmental conditions. The transformation process of naturally competent cells involves three basic steps: binding, uptake, and recombination of DNA into the chromosome of the recipient cell via homologous recombination. Natural competence is a physiologically and genetically determined property of a particular bacterial strain.

Artificial competence is not encoded in the cell's genes. In this case, the cells are made permeable to extracellular DNA using conditions that do not normally occur in nature. Specifically, artificial competence results from the treatment of noncompetent cell cultures with chemical and physical agents to permit the uptake of extracellular DNA. Many established procedures are used to achieve artificial competence, and can be used to genetically engineer cells.

One method of achieving artificial competence in bacteria involves chilling cells in the presence of ionic solutions such as CaCl₂. This medium prepares the walls and membranes of cells to become permeable to extracellular DNA. The cell membrane is permeable to chloride ions, but is non-permeable to calcium ions. As the chloride ions enter the cell, water molecules accompany the charged particle. This influx of water causes the cells to swell and is necessary for the uptake of DNA. The cells are incubated with the DNA and then briefly heat shocked (42° C.), making the exterior of the cell even more permeable, allowing the DNA to enter the cell. A major disadvantage of the process is that some cells can die after being exposed to heat shock.

Another method of achieving artificial competence in cells is electroporation. Electroporation is used to introduce gaps in the cell walls and membranes by briefly shocking them with an electric field of 100-200V. Extracellular DNA can then readily enter the cell through these holes. Shortly after electroporation, natural membrane-repair mechanisms in cells close these holes in the cell's exterior membranes. The disadvantage of this method is that lengthy or excessively high voltage pulses can lead to the death of most cells in the culture. Electroporation used for transformation also has limited efficiency due to arcing and variability among different laboratories and species.

Yet another method of artificial competence can be conducted with polyethylene glycol (PEG)-mediated protoplast fusion. In this method, protoplasts are used as carriers to transfer foreign genes into new organisms. A protoplast is usually isolated from a cell and inserted into another selected cell in a process called protoplast fusion. The most common chemical used to stimulate protoplast fusion is PEG, which increases the permeability of cell membranes. The disadvantage of using this method is its unpredictability because it cannot be used on some species, and the chemical fusion frequency may vary for a given species. This method is also labor intensive and technically demanding because of the fragility of protoplasts.

Therefore, there remains a need for a method for a high efficiency DNA transformation protocol for non-naturally transformable cells.

### SUMMARY

Aspects of the invention generally provide a method for improving the ability of non-naturally transformable cells to take up and integrate extracellular DNA. In one aspect, the invention provides a method for transforming cells, comprising placing, in a vessel, a mixture of deoxyribonucleic acid (DNA) and a recipient cell culture prepared for the uptake of the DNA, injecting a supercritical fluid into the vessel, removing the recipient cells from the vessel, and placing removed cells into a growth media with selective conditions to allow expression of transformed DNA.

In another aspect, the invention provides a method for transforming cells, comprising placing, in a vessel, a mixture of extracellular DNA and a recipient cell culture prepared for the uptake of the extracellular DNA, injecting a supercritical fluid into the vessel, injecting one or more inert gases into the vessel, removing the recipient cells from the vessel, and placing removed cells into a growth media with selective conditions to allow expression of transformed DNA.

### BRIEF DESCRIPTION OF THE DRAWINGS

For a further understanding of the nature and objects of the present invention, reference should be made to the following detailed description, taken in conjunction with the accompanying drawings, in which like elements are given the same or analogous reference numbers and wherein:

FIG. 1 exhibits the main processing steps entailed by the embodiments of the invention.

FIG. 2 shows the apparatus according to one embodiment of the invention.

### DESCRIPTION OF PREFERRED EMBODIMENTS

The words and phrases used herein should be given their ordinary and customary meaning in the art by one skilled in the art unless otherwise further defined.

Artificial competence results from the treatment of noncompetent cell cultures with chemical and physical agents to permit the uptake of extracellular DNA. As described earlier, the conventional methods used to achieve artificial competence entail many problems. The use of electroporation, heat shock, and protoplasts all have limited efficiency, are labor intensive, and compromise the viability of the fragile cells. Aspects of this invention can be used to develop a highly efficient protocol for achieving artificial competence in non-naturally transformable cells using supercritical fluids. High efficiency of transformation of DNA can be achieved by placing a DNA-cell culture mixture in a vessel containing a supercritical fluid, and carefully adjusting parameters in the vessel such as temperature and pressure of CO₂ and other gases.

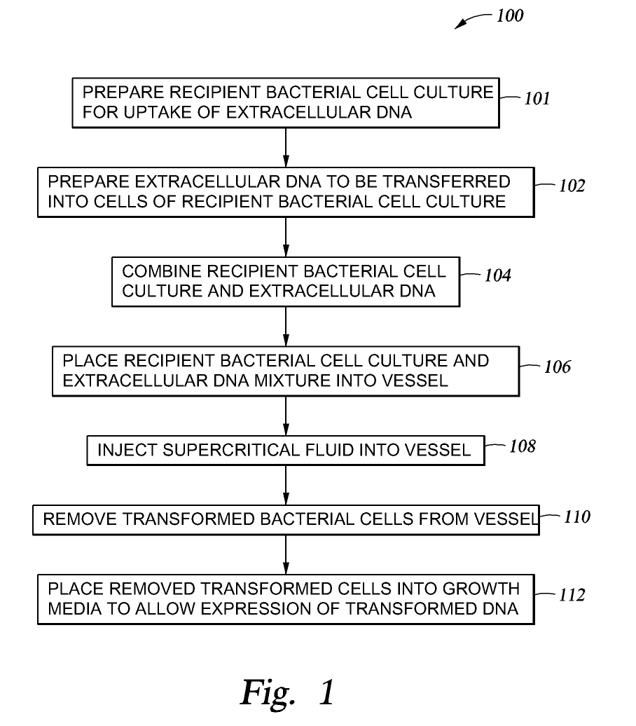

FIG. 1 is a flow diagram of a process 100, according to one embodiment of the present invention. The process 100 includes the preparation of a recipient bacterial cell culture for the uptake of extracellular DNA 101 and the preparation of the extracellular DNA to be transferred into bacterial cells of the recipient bacterial cell culture 102. The process 100 further includes the combination of the recipient bacterial cell culture and the extracellular DNA 104, placement of the DNA-cell culture mixture in a vessel 106, injection of one or more gases and supercritical fluids into the vessel 108, removal of transformed bacterial cells from the vessel 110, and placement of cells into a growth media with selective conditions to allow expression of transformed DNA 112.

The processing steps 101-112 according to the embodiments of the invention are described below. The embodiments described herein are provided to illustrate the invention and the particular embodiments shown should not be used to limit the scope of the invention.

The first processing step 101 according to FIG. 1 involves the preparation of a recipient bacterial cell culture for the uptake of extracellular DNA. A variety of bacterial strains can be chosen as a recipient strain during transformation, including strains of Lactobacillus spp., Enterococcus faecalis and Escherichia coli. To illustrate at least some embodiments of this invention, bacterial strains capable of artificial competence are preferred. E. coli is one preferred recipient strain because its genome is well documented and understood, and optimum growth conditions have been studied thoroughly. The embodiments described herein for preparing a bacterial cell culture for transformation are merely illustrative and should not be used to limit the scope of the invention, or the types of recipient cells used.

According to one embodiment, a method of preparation of a recipient bacterial culture used in processing step 101 follows a modified protocol of Chung, C. T., S. L. Nimiela, R. H. Miller; 1989; “One-step preparation of competent Escherichia coli: transformation and storage of bacterial cells in the same solution”; Proc. Natl. Acad. Sci; USA; 86: 2172-2175, herein incorporated by reference. E. coli cells are grown in LB (Luria-Bertani) broth (1% tryptone, 0.5% yeast extract, 0.5% NaCl) at 37° C. for 12-18 hours aerobically. The E. coli culture is then diluted (1:100) with LB broth and incubated at 28° C. with shaking in a mechanical shaker at 220 rpm. The culture is allowed to grow to the early exponential phase, which is typically reflected by an optical density of 0.5 to 0.6 when the absorbance of the culture is read in an ultraviolet spectrophotometer at 600 nm wavelength. The E. coli cells are then separated from the growth medium by centrifugation at 5000×g for 10 minutes at 4° C. The cells are in pellet form after this step, and the pellet is resuspended in a salt solution consisting of 25 mM MgCl₂ or CaCl₂ in distilled water at a pH between 6.5 and 7. This medium prepares the cell walls and cell membranes of bacterial cells to become permeable to plasmid DNA. The E. coli cells remain in this solution for about 10 minutes to about 1 hour until ready to be mixed with the transforming DNA. Other embodiments may include a resuspension solution consisting of TSS buffer, which includes LB broth with 10% polyethylene glycol (PEG), 5% dimethylsulfoxide (DMSO), and 50 mM Mg²⁺ (MgSO₄ or MgCl₂) at a final pH of 6.5.

The second processing step 102 according to FIG. 1 involves the preparation of the DNA to be transferred into recipient bacterial cell culture. A variety of methods for preparing DNA to be transferred into cells may be used. The DNA can be cloned and isolated in genomic form as replicated or “naked” DNA, which has the ability to enter bacterial cells and be incorporated into their genome. The DNA can also be isolated and prepared as double-stranded linear DNA and inserted into a bacteriophage, which can travel into a cell and release the foreign DNA. According to one embodiment, preparation of foreign DNA in a plasmid is contemplated. Plasmids contain a selectable marker (i.e., a gene that encodes for antibiotic resistance), an origin of replication (which is used by the DNA making machinery in the recipient bacteria as the starting point to make a copy of the plasmid) and a multiple cloning site. The multiple cloning site has many restriction enzyme sites and is used to insert the DNA of interest. The embodiments described herein are provided to illustrate the invention and the particular embodiment shown should not be used to limit the scope of the invention, or the types of methods used to prepare transforming DNA.

To prepare DNA to be transferred into cells of a recipient bacterial cell culture as in processing step 102, cell membranes must be broken open in a process known as lysis, and the chromosomal DNA is recovered from the resulting lysate. The DNA of interest is then purified to ensure the DNA is separated from proteins and ribonucleic acid (RNA). The extracted DNA is also quantified to verify that enough DNA was isolated for transformation. The concentration of DNA samples can be determined by measuring the absorbance of the DNA solutions at a specific wavelength. The amount of light absorbed by a DNA solution is directly related to the concentration of DNA in the solution. To measure the amount of light absorbed by a DNA sample, a spectrophotometer can be used in one embodiment. A spectrophotometer is an instrument that shines a light beam of a specific wavelength through the sample, and measures the amount of light that is absorbed by the solution.

Processing step 102 also involves the characterization of the DNA to be transformed. In one embodiment, the DNA molecules can be characterized by cutting the DNA with special enzymes known as restriction endonucleases, or restriction enzymes. These enzymes recognize specific base sequences in DNA that are called restriction enzyme recognition sites. Each restriction enzyme has its own recognition site. A restriction digest reaction is then conducted, which involves a mixture of the DNA to be analyzed with the restriction enzyme(s) that are used, in the presence of an appropriate buffer. Different restriction enzymes perform best at different pH and salt conditions. Commercially available restriction enzymes come with special buffers that are carefully optimized for maximum enzyme activity. The DNA fragments that are generated are then separated by size by electrophoresis of the DNA pieces through an agarose gel. Then, with the help of another enzyme DNA ligase which joins cut DNA ends together, different DNA fragments can be cut and pasted to give new combinations called recombinants.

Plasmids comprise one possible genetic structure to be transferred into E. coli cells of a recipient cell culture in processing step 102. Plasmids are circular double-stranded DNA molecules separate from the chromosomal DNA and capable of autonomous replication. Restriction enzymes can be used to cut open a plasmid in order to insert the extracellular DNA that will be transferred. The extracellular DNA to be inserted gene will have to be cut with the same restriction enzymes, so as to give it ends that will match up with the cut ends of the plasmid. The extracellular DNA can then be inserted into the plasmid using the enzyme DNA ligase to seal the two DNA molecules together. Once this recombinant plasmid is constructed, it can be put back into a recipient E. coli cells by transformation. The extracellular DNA that has been inserted will be replicated every time the plasmid DNA is replicated in the recipient E. coli cells. The inserted DNA can also be expressed in the recipient E. coli cells, that is, it can be copied to make messenger ribonucleic acid (mRNA), and the mRNA can be translated by the recipient cell's machinery to make a protein encoded by the DNA.

A number of methods and commercially available kits can be used in the embodiments of processing step 102 to prepare and purify plasmid DNA for transformation. One embodiment includes the use of a Qiagen Plasmid Purification Kit (Qiagen, Inc., Germantown, Md.). This plasmid preparation protocol used in this embodiment is based on a modified alkaline lysis procedure, followed by binding of plasmid DNA to anion-exchange resin under appropriate low-salt and pH conditions. RNA, proteins, dyes, and low-molecular-weight impurities in the plasmid are removed by a medium-salt wash. Plasmid DNA is eluted in a high-salt buffer and then concentrated and desalted by isopropanol precipitation. The plasmid DNA is now ready to be mixed with the recipient E. coli bacterial culture.

According to one embodiment, the third processing step 104, fourth processing step 106, and fifth processing step 108 according to FIG. 1 involve combination of the recipient bacterial cell culture and the extracellular DNA, placement of the DNA-cell culture mixture in a vessel, and injection of supercritical fluids into the vessel, respectively. In one particular embodiment of processing step 104, a portion of E. coli cells (10-1000 μl) is mixed with 0.1 to 10 μg of plasmid DNA in a dialysis bag with a membrane (Spectra/Por 2, MWCO12000-14000, 15.9 mm diameter, available from Spectrum Laboratories, Rancho Dominguez, Calif.). The mixture is allowed to sit for about 1 minute to 1 hour, allowing an initial amount of plasmid to enter the CaCl₂-induced gaps in the E. coli cell membranes. Mixtures containing cells other than E. coli may be allowed to sit for time periods longer than 1 hour. Another embodiment includes polypropylene micro-centrifuge tubes that can be used in place of dialysis tubing, after making a few needle holes in the lid.

The dialysis bag containing the mixture of recipient bacterial cell culture and plasmid DNA is then placed into a supercritical fluid vessel in step 106. A supercritical fluid is a substance which, when subjected to particular set of pressures and temperatures, exhibits a specific set of characteristics. Within these critical temperature and critical pressure zones, the distinction between the gaseous and liquid phases disappear, and the substance can only be described as a fluid with a unique set of properties and characteristics related to compressibility, homogeneity, and solvency. A number of supercritical fluids with similar properties may be introduced into the vessel by means of an attached pump in processing step 108. For example, the supercritical fluids may be generated from gaseous CO₂, H₂, O₂, or N₂O, or the mixture of these at different ratios. In one embodiment described herein, supercritical carbon dioxide is used during the transformation of plasmid DNA into the recipient E. coli cell culture. Supercritical CO₂ takes on properties of both a gaseous and aqueous phase while inactivating a range of microorganisms.

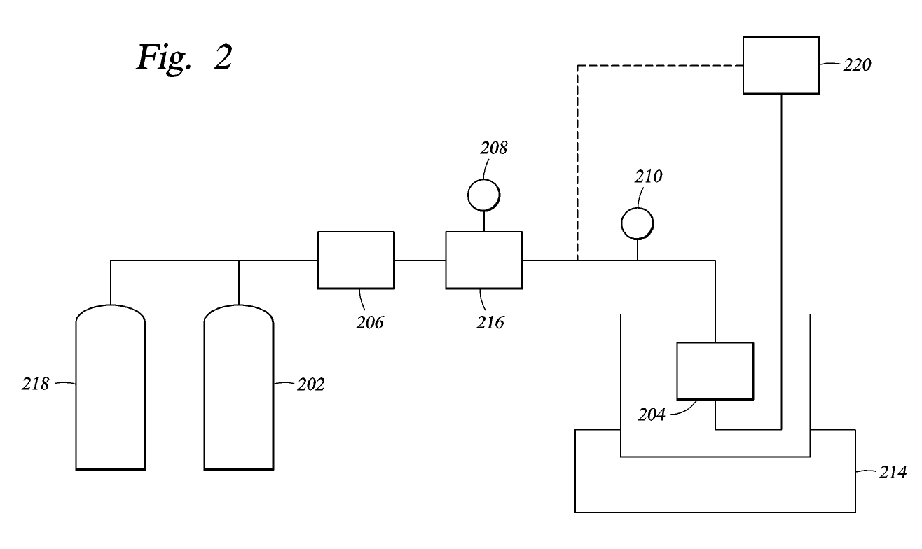

FIG. 2 illustrates an apparatus 200 used in one embodiment to expose the mixture of recipient bacterial cell culture and DNA to supercritical fluids, as described in processing steps 106-108 of FIG. 1. In this embodiment, gaseous CO₂ from a source 202 is compressed and heated before being transferred as a supercritical fluid to the vessel 204 containing the plasmid DNA and recipient E. coli cell culture mixture. Pressure adjustments can occur in several stages. As shown by pressure control 206, pressure is monitored at pressure gauge 208 and pressure gauge 210. A temperature control is exhibited by a water bath 214, which surrounds the vessel 204 to regulate the temperature of the vessel. Supercritical CO₂ is generated at pressures approaching 7.3 MPa, and temperatures above 31.1° C., according to one embodiment. Once supercritical CO₂ has entered the vessel by means of a pump 216, the operating pressure maintained within the vessel is between about 1100 psi and about 15,000 psi, according to one embodiment. Inert gases may be injected into the vessel from the source 218 to help tune and maintain the partial pressure and alter the properties of the injected supercritical CO₂. The inert gases that can be used include, for example, N₂, He, Ar, Kr, Xe, or Ne, or combinations thereof. When additional inert gases are added during treatment, the operating pressure in the vessel is maintained at a subsupercritical level for CO₂, between 500 to 15,000 psi. The operating temperature within the vessel during treatment of the recipient cells and DNA, according to one embodiment, is between about 1° C. and about 80° C., and the cooling temperature within the vessel after treatment is maintained between about 0° C. and about 40° C. In one embodiment, low-density CO₂ recovered from the vessel after treatment can be fed to a refrigerated condenser 220 and collected as a liquid. The liquid CO₂ can be recycled for the regeneration of supercritical CO₂.

In this embodiment, the plasmid DNA and recipient E. coli cell culture mixture remain in the vessel under supercritical conditions for up to 6 hours. Cell samples may be extracted after various residence times. For instance, samples can be removed from the dialysis bag after 1, 5, and 20 minutes of exposure to the supercritical fluid and then screened for the transforming DNA.

Referring again to FIG. 1, the sixth processing step 110 and seventh processing step 112 involve the removal of transformed bacterial cells from the vessel, and placement of cells into a growth media with selective conditions to allow expression of transformed DNA, respectively. After exposure to the supercritical fluid, the recipient E. coli cells in one embodiment are removed from the dialysis bag in step 110 and transferred into 1 ml LB broth with 20 mM glucose in step 112. The cells will be incubated at 37° C. with shaking for 1 hour to allow expression of the transformed DNA, more specifically, the newly added antibiotic resistance gene. After the phenotypic expression, the cells are diluted and plated on LB agar plates containing a selective antibiotic. The growth of transformed cultures is monitored after 24 hours of incubation at 25° C. to 37° C.

### EXAMPLE

In another embodiment, the strain Enterococcus faecalis is used as a transformation recipient. E. faecalis is grown for 12-18 hours in Brain Heart Infusion (BHI) medium in the presence of 5% CO₂. The culture is then diluted (1:10) in two 100 ml tubes, one supplemented with 3% (wt/vol) glycine and one without glycine, to an optical density of 0.3-0.4. The cells are then centrifuged for 10 min at 5,000×g at 4° C. The glycine-supplemented cells are resuspended in 1 ml of 0.25 M sucrose solution and glycine-deficient cells are resuspended in distilled water. The cells are mixed with a plasmid DNA which contains an antibiotic resistant gene that can be expressed in E. faecalis. The cells are then transformed in the supercritical fluid vessel as described above. The cells containing the transforming DNA of the plasmid are screened on BHI agar plates containing antibiotic after 24-48 hours of incubation at 37° C. For control experiments, E. faecalis cells that are not mixed with plasmid DNA are also plated on BHI agar plates containing the same antibiotic. To determine the viable cells before supercritical CO₂ treatment and survival of recipient cells after supercritical CO₂ treatment, LB and BHI agar plates are used without antibiotics. Transformation efficiency can then be calculated as transformants per μg of plasmid DNA used.

Preferred processes and apparatus for practicing the present invention have been described. It will be understood and readily apparent to the skilled artisan that many changes and modifications may be made to the above-described embodiments without departing from the spirit and the scope of the present invention. The foregoing is illustrative only and that other embodiments of the integrated processes and apparatus may be employed without departing from the true scope of the invention defined in the following claims.

1. A method for transforming cells, comprising:
a) placing, in a vessel, a mixture of deoxyribonucleic acid (DNA) and a recipient cell culture prepared for the uptake of the DNA;
b) injecting a supercritical fluid into the vessel;
c) removing the recipient cells from the vessel; and
d) placing removed cells into a growth media with selective conditions to allow expression of transformed DNA.

2. The method of claim 1, further comprising, prior to placing the mixture into the vessel:
preparing DNA to be transferred into cells of a recipient cell culture;
preparing the recipient cell culture for the uptake of DNA using buffers, ions, inorganic chemicals, organic chemicals, and combinations thereof; and
combining the DNA and the recipient cell culture.

3. The method of claim 2, comprising preparing the recipient cell culture for the uptake of DNA by suspending the cell culture in CaCl2.

4. The method of claim 2, comprising preparing the recipient cell culture for the uptake of extracellular DNA by suspending the cell culture in glycine.

5. The method of claim 2, comprising preparing the recipient cell culture for the uptake of extracellular DNA by suspending the cell culture in penicillin.

6. The method of claim 2, comprising preparing DNA to be transferred into cells of a recipient cell culture by insertion of the DNA into a plasmid followed by plasmid purification.

7. The method of claim 1, wherein the supercritical fluid can be generated from one or more of CO2, H2, O2, N2O, and combinations thereof.

8. The method of claim 1, wherein the operating pressure in the vessel is maintained at between about 500 and about 15,000 psi.

9. The method of claim 1, wherein the operating temperature in the vessel is maintained at between about 1° C. and about 80° C.

10. The method of claim 1, wherein the optional operating cooling temperature in the vessel is between about 0° C. and about 40° C.

11. A method for transforming cells, comprising:
a) placing, in a vessel, a mixture of extracellular DNA and a recipient cell culture prepared for the uptake of the extracellular DNA;
b) injecting a supercritical fluid into the vessel;
c) injecting one or more inert gases into the vessel;
d) removing the recipient cells from the vessel; and
e) placing removed cells into a growth media with selective conditions to allow expression of transformed DNA.

12. The method of claim 11, further comprising, prior to placing the mixture into the vessel:
preparing DNA to be transferred into cells of a recipient cell culture; preparing the recipient cell culture for the uptake of extracellular DNA using buffers, ions, inorganic chemicals, organic chemicals, and combinations thereof; and combining the extracellular DNA and the recipient cell culture.

13. The method of claim 11, wherein the inert gas is selected from the group consisting of N2, He, Ar, Kr, Xe, Ne, and combinations thereof.

14. The method of claim 12, comprising preparing the recipient cell cell culture for the uptake of extracellular DNA by suspending the cell culture in CaCl2.

15. The method of claim 12, comprising preparing the recipient cell culture for the uptake of extracellular DNA by suspending the cell culture in glycine.

16. The method of claim 12, comprising preparing the recipient cell culture for the uptake of extracellular DNA by suspending the cell culture in penicillin

17. The method of claim 12, comprising preparing DNA to be transferred into cells of a recipient cell culture by insertion of the DNA into a plasmid followed by plasmid purification.

18. The method of claim 11, wherein the supercritical fluid can be generated from one or more of CO2, H2, O2, N2O, and combinations thereof.

19. The method of claim 11, wherein the operating pressure in the vessel is maintained at between about 500 and about 15,000 psi.

20. The method of claim 11, wherein the operating temperature in the vessel is maintained at between about 1° C. and about 80° C.

21. The method of claim 11, wherein the optional operating cooling temperature in the vessel is between about 0° C. and about 40° C.
